(19) United States
(12) Patent Application Publication
(10) Pub. No.: US 2003/0216284 A1
(43) Pub. Date: Nov. 20, 2003

(54) Photoactivated drug therapy

(75) Inventors: Yoel Fink, Cambridge, MA (US); Edwin L. Thomas, Natick, MA (US); John D. Joannopoulos, Belmont, MA (US); James W. Winkelman, Brookline, MA (US)

Correspondence Address: Timothy J. Oyer, Ph.D., Wolf, Greenfield & Sacks, P.C., 600 Atlantic Avenue, Boston, MA, 02210, US

(73) Assignee: Massachusetts Institute of Technology, Cambridge, MA

(21) Appl. No.: 10/337,250

(22) Filed: Jan. 6, 2003

Related U.S. Application Data
(63) Continuation of application No. PCT/US01/41252, filed on Jul. 3, 2001.
(60) Provisional application No. 60/216,241, filed on Jul. 6, 2000.

(51) Int. Cl. A61K 31/00; A61N 1/30

(52) U.S. Cl. 514/1; 604/20

(57) ABSTRACT

A series of articles and techniques for controlled pharmaceutical delivery within a patient is described. An article includes at least one cavity having an interior dimension equal to a resonant mode of electromagnetic radiation to which the article is exposed. A standing wave is created within the cavity, causing a change in a diffusion characteristic of at least one component of the cavity, in turn causing release of a pharmaceutical from the cavity into an area of the body surrounding the article. Low-energy, non-destructive electromagnetic radiation, such as visible or near-infrared light, can be used.

### RELATED APPLICATIONS

This application is a continuation of PCT International Application No. PCT/US01/41252 filed Jul. 3, 2001, which was published under PCT Article 21(2) in English, and claims priority to U.S. application Ser. No. 60/216,241, filed Jul. 6, 2000. Both applications are hereby incorporated by reference.

### FIELD OF THE INVENTION

The present invention relates generally to pharmaceuticals, and more particularly to controlled-release pharmaceuticals activated by electromagnetic radiation that is not substantially absorbed by blood or tissue.

### BACKGROUND OF THE INVENTION

A preponderance of the current therapeutic treatment methods involves systemic administration of drugs for treatment of local disorders. This inherent characteristic of conventional drug delivery is the major cause of side effects. In addition it lowers the treatment success rate and increases the treatment cost.

The importance of controlled in-vivo delivery of therapeutic agents has been recognized. It is the subject of an intensive research effort in the US and abroad. A majority (an ultrasound mediated transdermal delivery system which offers temporal and spatial control was described by Mitragotri, S., Blankschtein, D., Langer, R., in Science 269 850 (1995) of the studies and devices that exist to date involve methods for temporal controlled release—also known as sustained release (Palik, E., “Handbook of optical constants of solid,” Academic Press, Vol. II, p. 1059-1077, (1988)). These methods typically include a matrix material which encapsulates the agent which has a twofold functionality. It provides a moisture and oxygen barrier and provides the mechanism for sustained release either by dissolution of the matrix or through a diffusion process. This mechanism provides a degree of control over the time dependence of the concentration of the drug in the blood stream, in cases where a system wide dispersion is required this control is advantageous. Potent therapeutic agents may have substantial system wide side effects which can be minimized if the release where to be directed to the target area. A highly localized release mechanism would allow for a high and effective concentration of drug and avoidance of system wide toxicity. A number of approaches have been explored in order to achieve localized drug delivery to non superficial locations, including ultrasonic transducers, phased arrays of antennas which cause local heating in deep body locations, and microchip drug delivery. Mitragotri, et al., Science, 269, 850 (1995) describe an ultrasound-mediated transdermal delivery system that offers temporal and spatial control.

While the above and other reports represent significant and useful contributions to the field of drug delivery, a need exists for low-cost, safe techniques for spatially-controlled release of pharmaceuticals within a patient, and components, compositions, and systems that facilitate this. It is one object of the invention to provide these.

### SUMMARY OF THE INVENTION

The present invention provides a series of methods and articles associated with drug delivery. Many of the methods and articles enable a drug delivery platform which releases the drug only in regions of need. In one embodiment, techniques of the invention involve photoactivation by low intensity light in the near visible portion of the spectrum.

In one aspect, the invention provides a series of methods involving drug delivery. One method involves selectively illuminating with visible or near infrared light, at a predetermined location within the body of a patient, an article comprising a pharmaceutical. The article is constructed and arranged to retain the pharmaceutical in a pharmaceutically inactive state in the absence of exposure to the visible or near-infrared light, while avoiding illumination of other like articles at locations within the body of the patient other than the predetermined location. The method further involves selectively activating the pharmaceutical via the exposure of the illuminated article to the visible or near-infrared light while avoiding activation of other, non-illuminated articles.

Another method of the invention involves selectively subjecting an article comprising a pharmaceutical, within the body of a patient, to conditions causing activation of the pharmaceutical while not subjecting body tissue or fluid surrounding the article to the conditions.

In another embodiment a method is provided involving exposing a solid article comprising a pharmaceutical to electromagnetic radiation, and causing activation of the pharmaceutical via the electromagnetic radiation.

In another embodiment a method involves subjecting a pharmaceutical within the body of a patient to physiologically-intolerable conditions, and causing activation of the pharmaceutical via the subjected conditions.

Another method of the invention involves activating a plurality of articles comprising pharmaceuticals within an area of a body of a patient by applying activation energy to the articles within the area, and essentially immediately terminating activation of the pharmaceuticals in the articles within the area by terminating the activation energy applied to the area.

Another method of the invention involves exposing a region of an article comprising a pharmaceutical to electromagnetic radiation incident upon the region, enhancing the energy density of the electromagnetic radiation selectively within the region of the article, relative to the energy density of the electromagnetic radiation incident upon the region of the article, and causing activation of the pharmaceutical via the electromagnetic radiation of enhanced energy density.

Another method of the invention involves exposing an article comprising material including a pharmaceutical to electromagnetic radiation below a threshold level of energy density, the threshold defined by a level of energy density required to cause activation of the pharmaceutical in the material independent structure, and causing activation of the pharmaceutical via electromagnetic radiation.

In another aspect the invention provides a series of articles. One article of the invention comprises a pharmaceutically-acceptable carrier including a region that enables the confinement of electromagnetic radiation, and a pharmaceutical associated with the region.

In anther embodiment the invention provides an article comprising a pharmaceutically-acceptable carrier constructed and arranged to allow activation of a pharmaceutical associated with the carrier under set conditions and to prevent activation of the pharmaceutical in the absence of the set conditions, wherein the set conditions are physiologically intollerable.

In another embodiment the invention provides an article comprising a pharmaceutical within a container including an arrangement of dielectric materials, the container including a photonic band gap and at least one defect state that allows for the existence for a localized electromagnetic mode.

In another embodiment the invention provides an article comprising an inner core region of a first material at least partially covered by an outer regions of a second material having a photonic band gap. The inner core region includes a pharmaceutical and a material capable of interacting with electromagnetic fields.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIG. 1 is a schematic illustration of a pharmaceutical container of the invention;

FIG. 2 is a schematic illustration of two electromagnetic radiation sources (lasers) the beams of which are crossed to define an interference volume;

FIG. 3 schematically illustrates a pharmaceutical article of the invention including a defect layer structure for break up and elimination from a patient; and

FIG. 4 schematically illustrates a photoactivatable pharmaceutical in accordance with the invention.

### DETAILED DESCRIPTION OF THE INVENTION

The present invention provides a series of methods and articles useful for spatially-controlled delivery of pharmaceuticals within an animal, specifically a human.

In one aspect, the invention provides a container for containing a pharmaceutical and delivery of the pharmaceutical within a patient. The container can make use of a release mechanism based on the creation of a high efficiency photon scavenging particle which dissipates a substantial portion of incident electromagnetic (EM) radiation energy in a very small volume culminating in the release of the active energy. By illuminating the particle with EM radiation of a particular frequency an interaction occurs leading to a change in the diffusion properties of the material and a subsequent release of a therapeutic agent.

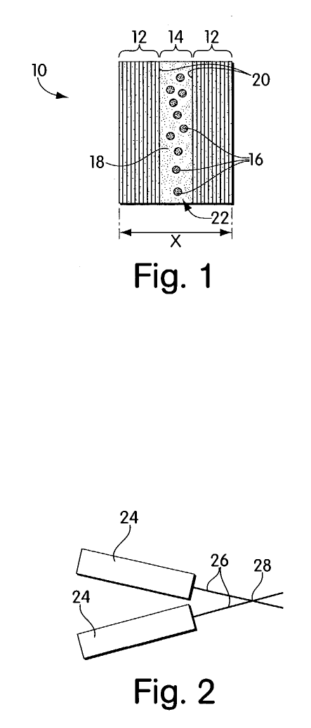

Referring now to FIG. 1, one embodiment of a pharmaceutical container 10 of the invention is illustrated schematically in cross section. Container 10 includes container walls 12 defining therebetween a container interior 14, or cavity. Cavity 14 contains a mixture of a pharmaceutical composition 16 within a binder, or matrix 18. Binder 18 containing pharmaceutical composition 16 is contained within, and typically fills, the interior of cavity 14 defined between interior surfaces 20 of walls 12. Interior cavity 14, as illustrated, is not isolated from the exterior of the container. Rather, an outlet 22 allows free communication between the interior of the cavity and the environment surrounding the article.

The components defining article 10 are constructed of a biologically-compatible material. This means that components defining the container are biodegradable and bioabsorbable, or can be readily eliminated from the body. Such components are well known, and can be easily selected by those of ordinary skill in the art using criteria described herein relating to the functional requirements of the various components.

Article 10 is constructed and arranged to contain pharmaceutical 16 within the article under one predetermined set of conditions, and to release pharmaceutical 16 to the environment surrounding the article under another set of conditions. One particularly useful set of conditions causing the release of pharmaceutical 16 is exposure to visible or near-infrared light at a frequency selected to cause release. Thus, the article is constructed to contain the pharmaceutical in the absence of exposure to this light, and to release the pharmaceutical upon exposure to a minimum quantum of the light. This can be accomplished where container 14 has an interior dimension equal to a resonant mode of the visible or near-infrared light.

Walls 12 can be constructed of a material such that interior surfaces 20 of the walls are highly-reflective, or perfectly-reflectively of the light and, where an interior dimension of the container (an interior dimension of section 14) is equal to a resonant mode of the light, then a standing wave, or resonance, can be established within cavity 14, heating binder 18 from a state in which it is viscous enough (essentially solid) to retain the pharmaceutical within the container to a state in which its viscosity drops, or it otherwise breaks down or changes state allowing release of pharmaceutical 16 through passage 22 and into the environment surrounding the article. Resonance is established within cavity 14 for a period of time sufficient to release the pharmaceutical, i.e. for a period of time sufficient to change a diffusion characteristics of binder 18 allowing the pharmaceutical to be released.

In a preferred set of embodiments, article 10 can be constructed from the following materials. Walls 12 can be constructed of any material that, upon exposure to electromagnetic radiation at a frequency that can cause resonance within cavity 14, will allow a standing wave to be defined within the cavity. Such materials are known to those of ordinary skill in the art and a particularly preferred material is described in international patent publication no. WO 98/35248 of Thomas, et al., entitled Polymeric Photonic Band Gap Materials, published Aug. 13, 1998 and incorporated herein by reference. Described in WO 98/35248 are polymeric materials including 1, 2, or 3 dimensional dielectric periodicity in structure of a dimension on the order of 100-1000 nm which define photonic band gap structures useful for optical elements in which certain frequencies of radiation are blocked, totally reflected, etc. Block copolymers can be synthesized and selected to self-assemble into such structures, as defined in Thomas, et al., and define one particularly useful set of starting materials. When used to define walls 12 of article 10, with the article exposed to electromagnetic radiation selected both to resonate within cavity 14 and to be highly or totally internally reflective within the cavity (reflected by interior walls 20), then a highly energetic standing wave can be formed within the cavity.

Pharmaceutical 16 can be essentially any agent desirably released within a body of a patient, particularly at a specific and predetermined location within a patient. A non-limiting, exemplary list of pharmaceuticals and conditions desirably treated using pharmaceuticals appears below. Binder 18 can be any material that, in the absence of exposure of the article to the electromagnetic radiation, retains pharmaceutical 16 within cavity 14 but, upon creation of a standing wave of the radiation within cavity 14, releases pharmaceutical 16 by undergoing a change in diffusion characteristic. Binder 18 can be a mixture of components, some of which undergo the change allowing release, and others that do not. It is important only that there be sufficient quantity of a selected material within cavity 14, no matter in what proportion or how distributed, that will allow release of the pharmaceutical upon creation of resonance within the cavity. Of course, binder 18 should satisfy other requirements such as physiological compatibility, as noted. In one convenient embodiment binder 18 is a single material that undergoes a rapid change in diffusion characteristic upon temperature change characteristic of creation of resonance within the cavity. For example, a polymeric material having a glass transition temperature higher than living body temperature, but easily attainable upon resonance of the radiation within the cavity for a period of time easily tolerable by physiology of the body through which the radiation passes. “Living body temperature” in this context means normal body temperature of the animal to which the treatment of the invention is administered, including slightly abnormal, but tolerable temperatures (e.g. fever).

Container 10 can be of essentially any shape, and in preferred embodiments (as illustrated) includes at least one opening 22 out of which pharmaceutical 16 can pass upon change in diffusion characteristic of binder 18. Any number of openings 22 can be provided, and the openings can be of any dimension allowing release of pharmaceutical 16. For example, where a pharmaceutical 16 defines individual small molecules, the structure of walls 12 may include naturally-occurring pores that are large enough to allow release of the small molecules, and separate passages 22 need not be provided for. In other embodiments, passages are not required at all. For example, in some embodiments a component of a wall 12, or walls 12 in their entirety, can be selected of material that will form openings upon thermal activation of material within cavity 14. For example, walls 12 can be constructed of material that will thermally degrade upon establishment of resonance within cavity 14, allowing release of pharmaceutical 16.

Article 10 is very small. Dimension x, as illustrated in FIG. 1, can be on the order of less than 10 microns. Preferably, article 10 is less than about 5 microns in dimension (its largest dimension), more preferably less than about 1 micron. In some embodiments the article can be less than about 0.1 microns, or about 0.3 microns in its largest dimension. The article can be administered internally of a patient in any manner, including orally, by injection, by intervention such as laparoscopy or catheterization, surgically, etc.

One significant advantage of the invention is that relatively low power, non-destructive electromagnetic radiation can be used to define a standing wave within cavity 14 causing release the pharmaceutical from the article. Specifically, light that is not readily absorbed by red blood cells or water, such as visible or near-infrared light, can be used. Light within the 0.65-1.3 micron wavelength is particularly preferred. Thus, a plurality of articles 10 can be introduced into the bloodstream and, at a desired treatment location, the electromagnetic radiation can be applied. For example, for treatment of a generalized area under the skin at a particular location the articles can be introduced into the bloodstream and light can be applied to that area of the body, causing release of pharmaceutical 16 at that area. For more precise and/or deeper body location release, crossed beams of electromagnetic radiation (as illustrated schematically in FIG. 2) can be used. Two or more beams can be arranged so as to intersect at a desired location, defining an interference volume. In this case the individual beams can be of low enough intensity so as not to cause sufficient resonance within cavity 14 to release pharmaceutical but, at the intersection of the two or more beams, intensity is great enough to cause pharmaceutical release. Those of ordinary skill in the art of radiative chemotherapy and the like are well-acquainted with establishment of such intersecting beams of radiation. In FIG. 2 electromagnetic radiation sources 24 can define lasers, emitting laser beams 26 that intersect at location 28. As an example, sources 24 can be arranged, with respect to a patient, such that intersection location 28 is a tumor within the body desirably treated with pharmaceutical 16. Articles 10 located within the tumor will release their pharmaceutical 16, while articles randomly distributed at other locations (including locations within beams 26 but not at intersection 28) will not release pharmaceutical.

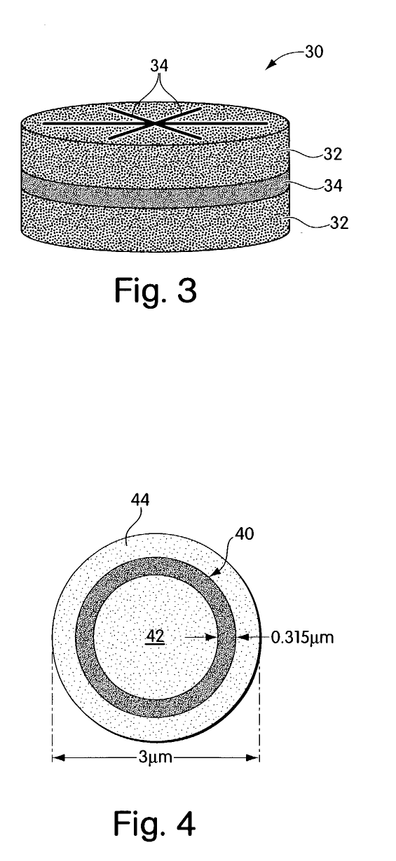

Referring now to FIG. 3, an article 30 of the invention is illustrated schematically which is fabricated to allow easy elimination from the body, where the article is large enough not to be easily eliminated when intact. Article 30 includes sections 32 divided by separating layers or portions 34 that separate sections 32 into individual component portions smaller than the whole of article 30. Portions 32 are multi-component portions that include pharmaceutical 16 (not illustrated) in binder 18 or other material that releases pharmaceutical 16 upon exposure to the electromagnetic radiation. Sections 34 are made of a biodegradable material that, upon a predetermined period of time within the body, will degrade, breaking up article 30 into smaller, intact portions of material 32 which can be easily eliminated from the body. Biodegradable polymers can be tailored to biodegrade over any of a variety of selected, predetermined periods of time, as is known to those of ordinary skill in the art. Accordingly, where article 30 is large enough that natural elimination from the body would be difficult, then after the radiation causing release of pharmaceutical from article 30 (and essentially break up of article 30) at a predetermined body location, non-irradiated articles 30 that remain randomly distributed within the body will break up into smaller components via biodegradation of material 34 over time. These smaller components, since not exposed to the electromagnetic radiation, will not release their pharmaceutical component. Instead, these intact, broken-up particles of pharmaceutical within binder will be naturally eliminated.

A more specific description of components and techniques, exemplary materials, and theory of operation, will now be presented. By utilizing a novel biocompatible optical microcavity design an efficient dissipation of electromagnetic energy in a very small volume is achieved which then leads to the drug release. Tailoring the resonant mode of the microcavity allows one to choose the activating light wavelength, in particular, light in the benign near-visible portion of the spectrum can be used. The microcavities are designed such that the release takes place only if the microparticle is illuminated by light of the correct frequency thus enabling highly localized targeted drug release. The materials used in the fabrication of the microcavities are FDA approved for use in in-vivo applications.

Potential applications are numerous; it can be used to deliver high concentrations of chemotherapeutic drugs to a targeted tumor while allowing virtually negligible of concentrations of drug elsewhere. This will substantially lower the systemic side effects that currently limit cancer chemotherapy. Other major applications of this system would include local delivery of antibiotics to infected tissue, anesthetics to subdural locations, or even the administration of narcotics to areas of acute pain.

The controlled release mechanism described below is actuated by low intensity electromagnetic radiation in the near visible (0.65-1.3 μm) spectrum and offers a high degree of temporal, spatial and angular control of the release characteristics.

Our ability to actuate by low intensity near visible light allows for portable low cost devices. In principle any treatment method of a local ailment is improvable by our approach. Potential applications are numerous; it can be used to deliver high concentrations of chemotherapeutic drugs to targeted tumor while allowing virtually negligible of concentrations of drug elsewhere. This will substantially lower the systemic side effects that currently limit cancer chemotherapy. Other major applications of this system would include local delivery of anesthetics to subdural locations, or even the administration of narcotics to areas of acute pain.

The first is a micron size particle made of a photonic crystal which is a structure with a periodic variation in its dielectric function. In the simplest case the photonic crystal is made of multilayers of alternating dielectric constant materials. In the midst of the photonic crystal structure is a cavity regime defined by a spatial extent or optical character which is different than the layers. This regime can be also seen as a defect in the otherwise regular periodic structure. The cavity is filled with a light absorbing material (absorber), with a material which changes its diffusion properties upon heating (gate) and with the therapeutic agent.

The second component is a laser source (with the possibility of multiple sources) emitting light at a wavelength for which the body is transparent. The penetration depth of the incident light is affected by two primary mechanisms: absorption and scattering. By operating in the 0.65-1.3 μm spectral range the absorption losses are minimized since both the red blood cells and water (k_{H}_{₂}_{O}(λ=1.301 μm)=1.861×10⁻⁵,k_{H}_{₂}_{O(λ=0.65} μm)=1.674×10⁻⁸,have little absorption in this range (Palik, E., “Handbook of optical constants of solid,” Academic Press, Vol. II, p. 1059-1077, (1988)), scattering is the dominant factor that effects the penetration depth but the forward component of the scattering in this regime is substantial.

By tuning the optical length of the microcavity it is possible to create resonant electromagnetic modes of specific frequency and k vector which are localized in the defect region. The laser source emits light corresponding to the cavities resonant frequency, the overlap region of the laser beam and the vessel containing the particles (blood vessel) defines an activation volume. When the cavity passes through the activation volume a large electromagnetic field density is built up inside the defect layer. The binding layer contains a material which has an imaginary index of refraction at the resonant frequency. The energy concentrated in the defect regime is dissipated through absorption causing an increase in the layer temperature leading to changes in its diffusion properties (undergoes a phase transition or goes through a glass transition) this results in the release of the therapeutic agent.

Characteristics of the Microcavity Based Release Method

1. Depending on the application and administration method (oral or by injection) the drug loaded microcavities can be dispersed throughout the blood stream or can be concentrated locally in a tissue. The drug is released only after the microcavity is illuminated by an electromagnetic wave of a certain frequency, direction and intensity.

2. The spatial resolution of the release region is defined by the activation volume which is the region where the laser beam (or beams) and the vessel containing the particles overlap. A control over the extent of this volume can be achieved by crossing two laser beams and by collimating the beams. This approach would entirely avoid release of the drug along the pathlength of the beam or anywhere else in the body except for the target.

3. The dissipation of the electromagnetic energy in the defect regime depends on the quality factor of the cavity, and the absorption coefficient of the absorber. High quality factor cavities will increase the conversion efficiency of electromagnetic energy to heat allowing use of low power lasers.

4. The rate of release of a particular microcavity can be tailored by the choice of the gate material. Materials which have a dramatic change in their diffusion properties upon heating will tend to release the drug quickly.

5. Control over the rate of release can be achieved by the intensity of the incident light.

6. The frequency of the laser is chosen such that a minimal fraction of the power is dissipated in the non targeted tissue. Depending on the depth of the targeted region different wavelengths may be selected according to their absorption in the tissue i.e for large penetration depths very small absorption of the laser in non targeted tissue can be tolerated.

7. The heating is extremely localized (˜0.3 μm) since the dissipation the EM energy is in an ultra thin layer (fractions of a micron), this is to be contrasted with microwave heating techniques where a large ˜1 cm³ is heated causing potential damage to non malignant tissue.

8. The small dissipation volume allows for the use of low incident power sources, thus minimizing potential radiation damage.

9. There is a large flexibility in the choice of the illumination frequency, one can choose to work with benign frequencies such as the visible or near IR.

10. The sophistication of the device is primarily in the microparticle design. The light source can be relatively low cost portable and could potentially be a multi-frequency source.

11. Release of multiple agents at one site can be achieved by designing a structure that has multiple cavities each excited by a different frequency and control via use of sources of different frequencies a particular sequence of release. This method can be used to release two pro-drugs A and B which need to react in order to create C which is the active drug molecule.

12. Another possibility is the use of multiple resonant cavities or cavities with different resonant modes to achieve a certain sequence of release such that a time dependent treatment procedure at short time intervals will be possible.

The objective of this section is to provide guidelines to the design of the activating light source. The microcavity release mechanism is activated by the absorption of photons in the cavity regime. This requires a minimal number of successful collision events between the microcavity and a photon of the right modal characteristics within a prescribed time interval. A successful collision is defined as a collision which leads to the photon absorption in the defect layer. The modal characteristics of the photon are those corresponding to the resonant defect mode. Furthermore the spatial localization characteristics depend on the ability to control the location of the successful collision events.

The first requirement of the source is that it emits light at a frequency which corresponds to the resonant condition of the microcavity.

The propagation of light in tissue is affected by absorption and scattering, the consequences of these two mechanisms on the nature of propagating photons is different. Isotropic absorption predominantly effects the number of propagating photons while leaving their modal characteristics unchanged. Elastic scattering changes the modal characteristics (i.e. k vector) of the propagating photons but leaves their total number constant. In order to maximize the number of photons (of specific character) propagating in the tissue one can devise a number of strategies.

Absorption of water and biological chromophores such as hemoglobin present in tissue is minimal in the 700-900 nm spectral regime. Photon migration in this regime is dominated by scattering of photons by micron size optical heterogeneous particles. The transport properties of photons in this spectral regime can be approximated by a diffusion model (O'Leary, M. A., Boas, D. A., Chance, B., Yodh, A. G., “Refraction of diffuse photon density waves,” Physical Review Letters, 69, 18, 2658-2661 (1992)). The exact propagation pattern is highly sensitive to the nature and number of the interfaces present in the tissue, in any case it is reasonable to assume that there is a diffuse photon density front which has an hemispherical shape. It is also reasonable to assume that the probability of finding a photon has a k dependency

P(\vec{r})>P(\vec{r})∀i≠0

where P\vec{r} is the probability of finding a photon at position \vec{r}, and \vec{r}₀=r\vec{k}₀, with \vec{k}₀ being the original propagation direction.

A consequence of the hemispherical propagation front assumption is that the number of photons decreases as the inverse distance from the source squared (where the source is defined as the point where the laser beam enters the tissue. By positioning a number of sources at different locations from the tumor a volume of maximum intensity is defined by the crossing of the hemispheres where the spatial and position of the volume is defined by the spatial separation of the sources (as defined above).

The light source design could include physical configurations of one or more LED's that can be adjusted by a physician or the patient himself depending upon the application. A flexible strap or pad with LED's emitting at one or more frequencies and positioned at different spatial separations thus forming a line, 2D, or even a 3D array of sources could be placed, for example, over a small or large painful joint in order to deliver analgesia, or around the circumference of the base of the penile shaft for treatment of erectile dysfunction.

The microcavity structure increases the probability of absorption by increasing the time certain photons spend in the defect regime. One can distinguish between three functionally distinct components in the defect which in reality could be achieved by one or more actual materials.

First a periodic structure with a defect of prescribed dimensions must be constructed such that a localized EM mode could exist in the defect regime. Since the microcavity will be interacting with a diffuse photon gas of no particular coherency characterized by a broad spread of the propagation vector it is important to design a resonant defect mode which has a weak k vector dependency. This will increase the number of photons which will interact and be absorbed by the microcavity. In addition a high quality factor of the microcavity will increase the absorption probability and the capture cross section for the photons. The periodic structure can be made of biocompatible materials which can be even degraded or metabolized by the body.

The second functionality is an absorbing capability, here a material which has a large absorption coefficient for photons corresponding to the defect modes is needed. The larger the absorption coefficient the higher the probability for absorption will be hence increasing the conversion efficiency of the EM to thermal energy.

The third functional component is a medium which changes its diffusion properties upon a temperature change at the normal body temperature the drug should have a very low diffusion in the medium while at elevated temperatures high diffusion is favorable. One class of materials which are known to have dramatic changes in their diffusion properties upon temperature changes are gels.

In the previous sections an outline of an absorption interaction between the photon and the material in the defect regime was presented. In general the objective is to cause a dramatic change in the properties of the defect material. Other interactions are also possible certain gels are known to interact with electromagnetic fields. Thus the absorption interaction was brought as an illustrative example. The methodology of increasing the interaction cross section by use of a microcavity is general and does not depend on the interaction type.

There are a number of possible methods for removing the unreleased portion of the drug;

One approach could involve the metabolism of the optical confinement structure (i.e. the photonic crystal) which could be made of materials which can be metabolized. The drug containing layer (˜0.2 μm) will then break into smaller pieces and be removed by the kidneys. To facilitate this process the drug containing layer could be patterned during fabrication and contain regions which are degradable (in black)and do not contain the drug but serve to buffer smaller drug containing regions (in gray) as shown in the figure below.

After the periodic structure dissolves the soluble regimes are exposed to the blood stream and are dissolved leaving much smaller insoluble drug containing particles which can be removed by virtue of their small size.

This system is virtually universally applicable to all pharmacologic therapies in which localized delivery of active agents is superior to simply having active drug circulate everywhere in the body in equal concentrations.

Examples of applications in which localized drug delivery will produce a medically significant advantage are numerous. In fact virtually the entire pharmacopea can be rethought with this potential advantage and very few drug treatments fail to be improved upon by localized delivery.

Among the major categories of drugs(see attached PDR 2000) the following categories with high likelihood of additional benefit from localized delivery are as follows:

A. Analgesics—localized relief of pain based on either focussed or diffuse release. For example, very small areas of joint pain, longer areas like a shoulder hip or knee could be irradiated. When a larger area such as a major joint is the target a device that does not need precise focussing that would be portable or suitable for home use.

B. Anesthetics—Localized anaesthetic would be particularly effectively improved by the new system. For example epidural anaesthetic during child birth could be achieved without introduction of a needle into the lower spinal region, i.e. non-invasively.

C. Central nervous system applications to particular region of the brain stem or cerebral cortex could represent an entirely novel approach to anesthesia with potentially very great advantages in the conduct of general surgery.

D. Anti-infective agents—many antibiotics are needed only in foci of infections. But currently must be delivered to the entire body in high concentrations. Examples of improved treatment of localized lesions would include abscesses of any organ, bowel infections, tuberculosis or any cryptic infections.

E. Antiparkinsonian—Particularly with very expensive agents that are currently administered systemically in high dose, a system for local delivery to selected portions of the brain for particular neurological diseases would represent an improvement in practice. The idea of focal delivery within the brain raises the prospect of entirely new approaches to both organic functional (psychiatric) disease. For a further example the possibility of appetite suppression or stimulation may be possible.

F. Bone metabolism—existing or future agents that could be useful in fracture repair, or repair of other defects of bone or prophylaxis against bone at risk of fracture from osteoporosis or other causes would benefit from localized delivery.

G. Cardiovascular agents.

H. Central nervous system stimulation, depressants and modifiers. This could even apply to treatment of sleep disorder.

I. Contraception.

J. Erectile dysfunction therapy—current management has many disadvantages in particular systemic administration have lethal side effects on patients with heart disease.

K. Gout

L. Hormonal disorder

M. Opthalmological treatments

N. Otic preparation

O. Skin and mucosal membrane

P. Psychotherapeutic agents

Q. Prostate disorders

The above listing is intended to be illustrative only and it is not meant to exclude other organs or disorders in which localized drug delivery would offer advantages over systemic delivery. A further consideration that is not meant to be exclusionary according to the above listing is the type of molecule that could be locally released. Currently used drugs and future drugs of virtually any type could be incorporated. These could be inorganic naturally occurring or synthetic molecules biologicals including proteins of any kind such as insulin for glucose level control as well as growth hormones, antibodies or even replacement or substitution molecules of amino acid nucleic acid or any other biologically useful type.

Yet another consideration is the possibility of route of administration. The novel formulation may achieve availability to the target site whether administered by inoculation into the blood stream, subcutaneously, directly into tissue in some particular region. Also meant to be included is direct absorption through the mucosa of the gastrointestinal track from either per-oral or per rectal administration or through the mucosa of the respiratory track.

The function and advantage of these and other embodiments of the present invention will be more fully understood from the examples below. The following examples are intended to illustrate the benefits of the present invention, but do not exemplify the full scope of the invention.

### EXAMPLE 1 (PROPHETIC)

### Fabrication of Photoactivatable Pharmaceutical

In this example use will be made of the maximum absorption efficiency criteria established in appendix A. With reference to FIG. 4, A 0.315 μm layer, 40, of a Poly (dl-lactide) (PLA) (n=1.5) with a 10 volume % of platinum particles (n=2.92, k=5.07@820 nm) achieves high absorption efficiency where >80% of the incident power is absorbed by the layer. An incident power of 50 mW will lead to a temperature increase of ˜300C per second in the layer. The glass transition temperature of PLA (amorphous) is approximately 55° C.

The Pt containing layer 40 is deposited on a porous PLA microsphere 42 which is subsequently coated with an additional layer 44 of porous PLA or Poly(glycolic acid) (PLGA). The total particle diameter should be approximately 3 μm. See FIG. 4.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described.

[image]

[image]

[image]

[image]

[image]

[image]

[image]

[image]

[image]

[image]

[image]

1. A method comprising:
selectively illuminating with visible or near-infrared light, at a predetermined location within the body of a patient, an article comprising a pharmaceutical, the article constructed and arranged to retain the pharmaceutical in a pharmaceutically inactive state in the absence of exposure to the visible or near-infrared light, while avoiding illumination of other like articles at locations within the body of the patient other than the predetermined location; and
selectively activating the pharmaceutical via the exposure of the illuminated article to the visible or near-infrared light while avoiding activation of the other, non-illuminated articles.

2. A method comprising:
selectively subjecting an article comprising a pharmaceutical, within the body of a patient, to conditions causing activation of the pharmaceutical, while not subjecting body tissue or fluid surrounding the article to the conditions.

3. A method comprising:
exposing a solid article comprising a pharmaceutical to electromagnetic radiation; and
causing activation of the pharmaceutical via the electromagnetic radiation.

4. A method comprising:
subjecting a pharmaceutical within a body of a patient to physiologically-intolerable conditions; and
causing activation of the pharmaceutical via the subjected conditions.

5. A method comprising:
activating a plurality of articles comprising pharmaceuticals within an area of a body of a patient by applying activation energy to the articles within the area; and
essentially immediately terminating activation of the pharmaceuticals in the articles within the area by terminating the activation energy applied to the area.

6. A method comprising:
exposing a region of an article comprising a pharmaceutical to electromagnetic radiation incident upon the region;
enhancing the energy density of the electromagnetic radiation selectively within the region of the article relative to the energy density of the electromagnetic radiation incident upon the region of the article; and
causing activation of the pharmaceutical via the electromagnetic radiation of enhanced energy density.

7. A method comprising:
exposing an article comprising material including a pharmaceutical to electromagnetic radiation below a threshold level of energy density, the threshold defined by a level of energy density required to cause activation of the pharmaceutical in the material independent of structure; and
causing activation of the pharmaceutical via the electromagnetic radiation.

8. An article comprising:
a pharmaceutically-acceptable carrier including a region that enables the confinement of electromagnetic radiation; and
a pharmaceutical associated with the region.

9. An article comprising:
a pharmaceutically-acceptable carrier constructed and arranged to allow activation of a pharmaceutical associated with the carrier under set conditions and to prevent activation of the pharmaceutical in the absence of the set conditions, wherein the set conditions are physiologically intolerable.

10. An article comprising:
a pharmaceutical within a container including an arrangement of dielectric materials, the container including a photonic band gap and at least one defect state that allows for the existence of a localized electromagnetic mode.

11. An article comprising:
an inner core region of a first material at least partially covered by an outer region of a second material having a photonic band gap, wherein the inner core region includes a pharmaceutical and a material capable of interacting with electromagnetic fields.

12. An article as in claim 8, comprising, within the region, the pharmaceutical, an absorber of the electromagnetic radiation, and a binder able to exist in a first state retaining the pharmaceutical within the region and a second state allowing release of the pharmaceutical from the region.

13. An article as in claim 12, wherein the binder and the absorber of electromagnetic radiation are the same material.

14. A method as in claim 12, wherein the binder and the absorber of electromagnetic radiation are different materials.

15. A method as in claim 7, wherein the pharmaceutical is confined within a region also including a binder able to exist in a first state retaining the pharmaceutical within the region and a second state allowing release of the pharmaceutical from the region and an absorber of the electromagnetic radiation.

16. A method as in claim 15, wherein the binder and the absorber of electromagnetic radiation comprise different materials.

17. A method as in any preceding claim, the conditions causing activation of the pharmaceutical comprising heat.

18. A method or article as in any preceding claim, further comprising a source of the electromagnetic radiation that is adaptable to the anatomy of a patient.

19. A method or article as in any preceding claim, wherein the activation involves release of the pharmaceutical.

20. A method as in claim 6 comprising:
exposing a container of a pharmaceutical to electromagnetic energy having a resonant mode at a dimension of the interior of the container thereby creating resonance within the container for a period of time sufficient to change a diffusion characteristic of at least one component of the container from a state maintaining the pharmaceutical within the container to a state allowing the pharmaceutical to be released from the container.

21. A method or article as in any preceding claim, wherein the activation involves a chemical or physical reaction caused by the electromagnetic radiation which, in turn, triggers a secondary chemical or physical reaction causing activation of the pharmaceutical.

22. An article as in claim 8, comprising:
a pharmaceutical within a container having an interior dimension equal to a resonant mode of visible or near-infrared light.

23. A method as in claim 1, wherein the article is administered orally to the patient.

24. A method as in claim 1, wherein the article is administered by injection to the patient.

25. A method as in claim 1, wherein the article has a maximum dimension of less than about 10 microns.

26. A method as in claim 1, wherein the article has a maximum dimension of less than about 5 microns.

27. A method as in claim 1, wherein the article has a maximum dimension of less than about 2 microns.

28. A method as in claim 1, wherein the article has a maximum dimension of less than about 1 micron.

29. A method as in claim 1, wherein the electromagnetic radiation is of low power.

30. An article as in claim 10, wherein the container includes interior walls that are highly reflective of the electromagnetic radiation.

31. An article as in claim 30, wherein the interior walls is entirely reflective of the electromagnetic radiation.

32. An article as in claim 10, wherein the container contains a binder with an imaginary index of refraction at a resonant frequency of the electromagnetic radiation.

33. An article as in claim 10, wherein the container contains a binder that is selected to undergo a change in diffusion characteristic upon exposure to resonance of the electromagnetic radiation.

34. A method or article as in any preceding claim, involving heating a binder within the container via the electromagnetic radiation, causing release of the pharmaceutical.

35. A method as in any preceding claim, comprising illuminating the article at the intersection of at least two beams of electromagnetic radiation.

36. A method as in any preceding claim, comprising illuminating the article with electromagnetic radiation that is not readily absorbed by blood or water.

37. A method as in claim 36, wherein the electromagnetic radiation is visible or near-infrared radiation.

38. A method or article as in claim 32, wherein the radiation is from about 0.65 to about 1.3 microns in wavelength.

39. A method or article as in any preceding claim, wherein the article contains more than one cavity.

40. A method or article as in any preceding claim, wherein the article at least two sections containing pharmaceutical separated by a biodegradable section.
